(19) United States
(12) Patent Application Publication
(10) Pub. No.: US 2025/0319286 A1
(43) Pub. Date: Oct. 16, 2025

(54) BALLOON CATHETER FOR DELIVERING A THERAPEUTIC AGENT TO VARIABLE LENGTH TREATMENT SITES

(71) Applicant: Bard Peripheral Vascular, Inc., Franklin Lakes, NJ (US)

(72) Inventor: Samuel M. Shreve, Scottsdale, AZ (US)

(21) Appl. No.: 18/861,017

(22) PCT Filed: Apr. 26, 2022
(86) PCT No.: PCT/US2022/026343

§ 371 Date: Oct. 28, 2024

(51) Int. Cl. A61M 25/10; A61M 25/00

(52) U.S. Cl. CPC A61M 25/1011; A61M 25/1002; A61M 25/104; A61M 2025/0006; A61M 2025/1015

(57) ABSTRACT

An apparatus for performing angioplasty on a lesion (L) and applying a therapeutic agent (A) thereto. The apparatus includes a catheter (10) having a first inflatable balloon (12a), such as a non-compliant balloon, adapted to compress the lesion when inflated. At least one port (18,18a) is provided to deliver the therapeutic agent to adjacent the lesion. Second (12b) and third (12c) inflatable balloons, which may be compliant balloons located distally and proximally of the first inflatable balloon for compressing the lesion, may be inflated to form a sealed chamber (C) including the compressed lesion and the therapeutic agent once delivered via the at least one port. The relative positions of the second and third inflatable balloons may be adjustable to accommodate different lengths of lesions.

### BACKGROUND OF THE INVENTION

Dilatation catheters with expandable elements or balloons are often used to treat lesions in vessels. In order to improve the treatment effect and, in particular, help to prevent restenosis, dilatation of a stenosed vessel is sometimes done in connection with the use of an applied therapeutic agent, or drug for short. However, in procedures where a drug-coated balloon is used and either there are multiple lesions, or a lack of correspondence between a balloon working surface and a treatment area, treatment of all would require multiple interventions each using a separate balloon. Furthermore, geographic misalignment, or “miss” is sometimes a concern, whereby a mismatch between the location of the drug on the balloon and the desired treatment area results, which can again lead to multiple interventions to achieve the desired treatment effect.

Others in the past have proposed arrangements by which a therapeutic agent may be applied independently of the balloon in order to overcome the above issues. A known arrangement uses two balloons arranged in series on a single catheter. The balloons are inflated on opposite sides of a desired location for treatment, such as a stenosis or lesion, in an effort to isolate the location.

While such an arrangement avoids the multiple intervention situation potentially required as a consequence of using a drug-coated balloon, it suffers from several limitations. For one, the balloons are fixed in position, thus eliminating the ability to customize the treatment area. This can limit the effectiveness of such a fixed balloon arrangement and, in the end, may realize little improvement over the use of a conventional drug-coated balloon. Furthermore, no means are provided for pressurizing the chamber thus formed to aid in delivery of the treatment agent, or for performing angioplasty therein.

Accordingly, a need is identified for an arrangement that overcomes some or all of the foregoing limitations, and perhaps others that have yet to be discovered.

### SUMMARY OF THE INVENTION

An object of the disclosure is to provide a balloon catheter that provides an improved treatment effect by delivering a therapeutic agent in a controlled manner to a variable length chamber including a selected portion of a vessel, and also allowing for performing angioplasty or increasing the pressure in the chamber during the procedure.

According to a first aspect of the disclosure, a medical apparatus for treating a lesion is provided. The apparatus comprises a shaft, a first inflatable balloon supported by the shaft, a second inflatable balloon supported by the shaft, a longitudinal position of the second inflatable balloon being adjustable relative to the first inflatable balloon, and a third inflatable balloon supported by the shaft between the first inflatable balloon and the second inflatable balloon.

In one embodiment, the shaft comprises an outer shaft supporting the first inflatable balloon and an inner shaft supporting the second inflatable balloon. The inner shaft further supports the third inflatable balloon. The inner shaft may comprise a first port proximal of the third inflatable balloon and a second port distal of the third inflatable balloon.

The outer shaft may comprise a retainer for retaining a position of the outer shaft relative to the inner shaft. The outer shaft may comprise a first inflation port for the first inflatable balloon, and the inner shaft comprises a second inflation port for the second inflatable balloon and a third inflation port for the third inflatable balloon. A proximal end of the shaft may comprise an infusion port for communicating with at least one opening in the shaft between the first inflatable balloon and the third inflatable balloon.

The first and second inflatable balloons may be compliant or semi-compliant, and may be generally spherical. The third inflatable balloon may be elongated, and may comprise a barrel section for compacting a lesion when inflated and tapered end sections connected to the shaft. The third inflatable balloon may be non-compliant.

According to a first aspect of the disclosure, a medical apparatus for treating a lesion is provided. The apparatus comprises a shaft including telescoping inner and outer portions. The inner portion of the shaft supports a first inflatable balloon. First and second inflatable balloons are supported by the inner portion of the shaft, a longitudinal position of the second inflatable balloon being adjustable relative to the first inflatable balloon by telescoping the outer portion of the shaft relative to the inner portion of the shaft. The inner shaft between the first inflatable balloon and the second inflatable balloon supports a third inflatable balloon.

In one embodiment, the inner portion of the shaft comprises a first port proximal of the third inflatable balloon and a second port distal of the third inflatable balloon. The outer portion of the shaft comprises a retainer for retaining the outer portion of the shaft relative to the inner portion of the shaft. The inner portion of the shaft comprises a first inflation port for the first inflatable balloon, the outer portion of the shaft comprises a second inflation port for the second inflatable balloon, and the inner portion of the shaft comprises a third inflation port for the third inflatable balloon. A proximal end of the shaft comprises a port for communicating with at least one opening in the shaft between the first and third inflatable balloons.

The first and second inflatable balloons may be compliant or semi-compliant, and may be generally spherical. The third inflatable balloon is elongated, and may comprise a barrel section for compacting a lesion when inflated and tapered end sections connected to the shaft. The third inflatable balloon may be non-compliant.

Yet a further aspect of the disclosure pertains to a medical apparatus for treating a lesion. The apparatus comprises a shaft including telescoping inner and outer portions, a first inflatable balloon supported by the inner portion of the shaft, and a second inflatable balloon supported by the inner portion of the shaft.

In one embodiment, the inner portion of the shaft comprises a first port proximal of the second inflatable balloon and a second port distal of the second inflatable balloon. The outer portion of the shaft comprises a retainer for retaining the outer portion of the shaft relative to the inner portion of the shaft.

The outer shaft may support a third inflatable balloon. The third inflatable balloon may be located proximally of the first and second inflatable balloons. The inner portion of the shaft comprises a first inflation port for the first inflatable balloon and a second inflation port for the second inflatable balloon, and the outer portion of the shaft comprises a third inflation port for the third inflatable balloon. A proximal end of the shaft comprises a port for communicating with at least one opening in the shaft between the first and second inflatable balloons.

The first inflatable balloon may be non-compliant and second inflatable balloons is compliant or semi-compliant. The first inflatable balloon may comprise a barrel section for compacting a lesion when inflated and tapered end sections connected to the shaft.

This disclosure also relates to a method for treating a lesion, comprising: (1) adjusting the position of a first inflatable balloon supported by a shaft relative to a second inflatable balloon supported by the shaft; (2) inflating a third inflatable balloon supported by the shaft between the first inflatable balloon and the second inflatable balloon. The method may further include the step of delivering a therapeutic agent to a space between the first and second inflatable balloons prior to the step of inflating the third inflatable balloon. The method may include the step of delivering a therapeutic agent to a space between the first and second inflatable balloons after the step of deflating the third inflatable balloon.

### BRIEF DESCRIPTION OF THE DRAWINGS

The above and further advantages of the present disclosure may be better understood by referring to the following description in conjunction with the accompanying drawings in which:

FIG. 1 is side schematic view of a balloon catheter according to one aspect of the disclosure;

FIG. 1A is a cross-sectional view of the balloon catheter of FIG. 1, taken along line 1A-1A of FIG. 1;

FIG. 1B is a cross-sectional view of a retainer associated with the balloon catheter, taken along line 1B-1B; and

FIGS. 2, 3, and 4 are progressive views illustrating one possible use of the balloon catheter.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the parts depicted in the drawings may be combined into a single function.

### DETAILED DESCRIPTION

In the following detailed description, numerous specific details are set forth to provide a thorough understanding of the presently disclosed invention(s). The disclosed embodiments may be practiced without these specific details. In other instances, well-known methods, procedures, components, or structures may not have been described in detail so as not to obscure the present inventive concepts.

The invention is not limited in its application to the details of construction and the arrangement of the components set forth in the following description or illustrated in the drawings. The inventive concepts disclosed are capable of other embodiments or of being practiced or carried out in various ways. Also, it is to be understood that the phraseology and terminology employed herein are for the purpose of description and should not be regarded as limiting.

Certain features of the disclosed embodiments that are, for clarity, described in the context of separate embodiments, may also be provided in combination in a single embodiment. Conversely, various features of the invention that are, for brevity, described in the context of a single embodiment, may also be provided separately or in any suitable sub-combination.

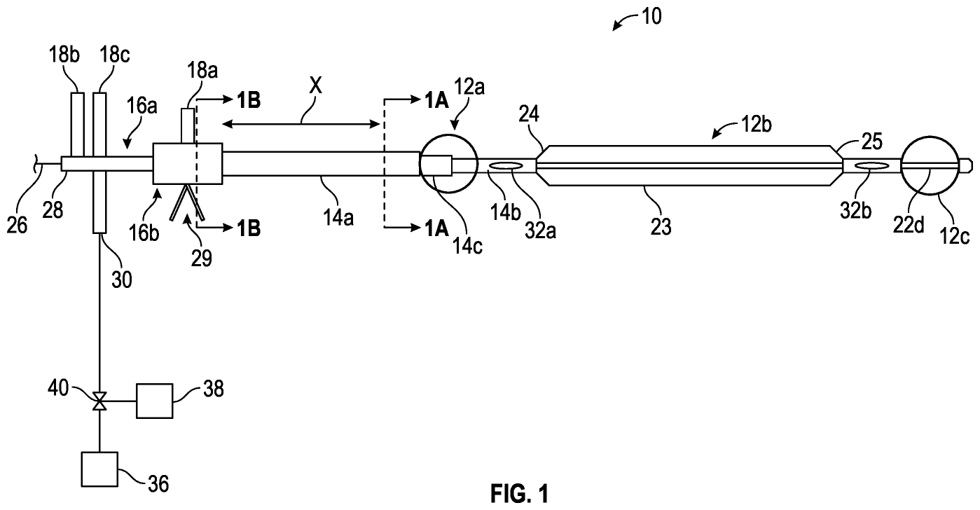

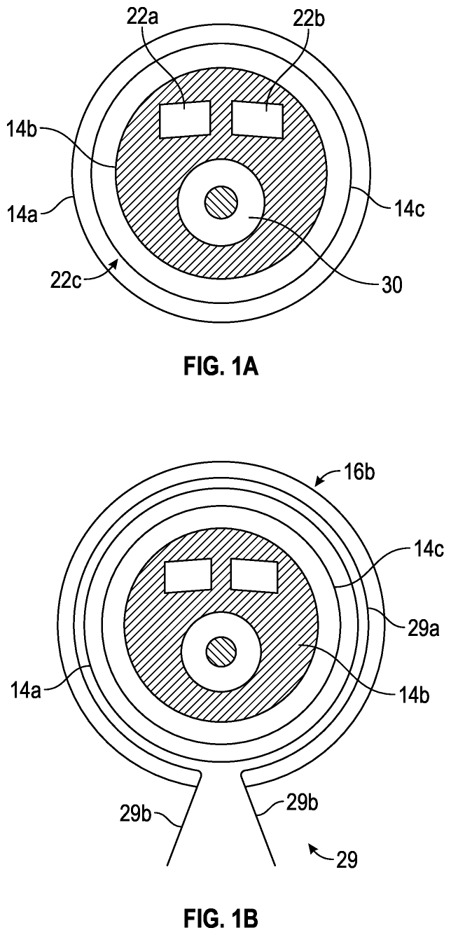

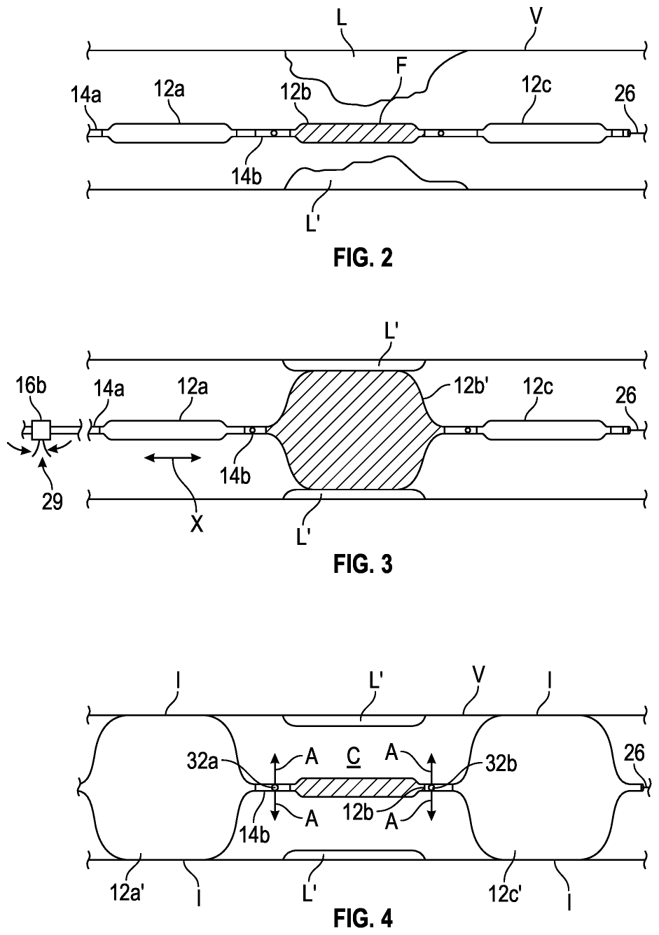

With reference to FIGS. 1 and 2, provided is a catheter 10 including a plurality of balloons 12 mounted on different portions of a telescoping catheter shaft 14. Specifically, the shaft includes an outer shaft or portion 14a supporting a first inflatable balloon 12a, such as located at a distal end thereof. An inner shaft or portion 14b of the shaft 14 is slidably received within the outer portion 14a. This inner shaft portion 14b supports a distal, third balloon 12c, such as at a distal end thereof, as well as an intermediate or middle balloon 12b. Relative movement among the shaft portions 14a, 14b allows for the spacing of the proximal balloon 12a relative to the middle and distal balloons 12b, 12c to be adjusted in a longitudinal direction.

Each balloon 12a, 12b, 12c may be independently inflatable via ports 18 associated with first and second hubs 16a, 16b at a proximal end of the catheter 10. Specifically, as perhaps best shown in FIG. 2, a first port 18a associated with the outer shaft 14a may be in fluid communication with the first balloon 12a via a first inflation lumen 22c (which as shown in FIG. 1A may be provided by an annulus between the inner shaft portion 14a and an intermediate tube 14c open to the interior compartment of the first balloon 12a). A second port 18b associated with the inner shaft 14b may be in fluid communication with the middle balloon 12b via a second inflation lumen 22b. Likewise, a third portion 18c also associated with the inner shaft 14b may be in fluid communication with the third or distal balloon 12c via a third inflation lumen 22c. Suitable indeflators (not shown) may be associated with each port 18a, 18b, 18c, or a single indeflator with suitable valving.

The first and third balloons 12a, 12c may be of similar construction, and generally spherical in shape, as illustrated, but could take other forms. In order to form a seal with the adjacent inner wall of the vessel when inflated, the first and third balloons 12a, 12c may be formed of a compliant or semi-compliant material. This means the balloons 12a, 12c have a degree of elasticity, and thus continue to expand with the addition of fluid pressure (which as noted above may be done independently to account for different portions of the vessel being contacted).

The second or middle balloon 12b may also be of the same construction. However, in the illustrated version, this balloon 12c is made noncompliant (meaning it does not further expand beyond a nominal diameter with added fluid pressure) and includes an intermediate portion 23, or “barrel,” and end portions 24, 25. In one embodiment, the end portions 24, 25 reduce in diameter or taper to join the intermediate portion 23 to the inner portion 14b of the catheter shaft 14 (and thus portions 24, 25 are generally termed cones or cone portions). This construction facilitates the performance of angioplasty by way of contact between a lesion and the barrel portion 23 of the balloon 12b when inflated.

Each balloon 12a, 12b, 12c can have any of a variety of diameters. For example, the diameters may range from 1.25-40 mm or 2.0-8.0 mm. The balloons 12a, 12b, 12c may have any of a variety of lengths, such as 10-300 mm or 20-300 mm. In the illustrated example, the first and third balloons 12a, 12c are larger in diameter and shorter than the second balloon 12b.

The shaft 14 also includes a guidewire lumen 22d formed by a tube that directs a guidewire 26 through the catheter 10. As illustrated in FIG. 2, this guidewire 26 may extend through the proximal end of the catheter 10 and through a port 28 of the hub 16a into the guidewire lumen 22d to achieve an “over the wire” (OTW) arrangement, but could also be provided in a “rapid exchange” (RX) configuration (in which the guidewire 26 exits a lateral opening closer to the distal end, not shown) or else is fed through the tip at a passage distally of the balloon 12 (“short” RX; not shown).

The second hub 16b including the inflation port 18a for the first balloon 12a connected to the outer portion 14a of the shaft 14 may also include a retainer 29. The retainer 29 serves to release the outer portion 14a of the shaft 14 for telescoping movement relative to (and along) the inner portion 14b. As shown in FIG. 1b, the retainer 29 may comprise a spring, such as a coil spring 29a, connected to a suitable gripper 29b for activating and relaxing the spring so as to allow for relative movement. Alternatively, the retainer 29 may comprise a clamp or collar with a locking mechanism to allow for selective release or retention to fix the relative position of shaft portions 14a, 14b.

According to a first aspect of the disclosure, the catheter 10 may be adapted to deliver a therapeutic agent or drug to a selected treatment location, independent of any balloon (that is, not as a coating on the balloon). This delivery may be achieved by providing the catheter 10 with a lumen 30 for delivering the therapeutic agent to one or more ports 32 formed in the shaft 14 and, in particular, the inner portion 14b to which the second and third balloons 12b, 12c are fixed. The one or more ports may be located proximal of the second balloon 12b, and/or may be distal of this balloon. In the version shown in FIG. 1, there are two ports 32a, 32b, one of which is proximal to the second balloon 12b, and the other of which is distal to it.

These ports 32a, 32b may serve to deliver therapeutic agents via infusion, as well as potentially to withdraw fluid. For this reason, a distal end of the lumen 30 may be attached to a source 36 of a therapeutic agent (such as, for example, a syringe including the selected agent, such as Paclitaxel, as one example). The lumen 30 may also be in communication with a source of vacuum 38 (which may be the same syringe delivering the therapeutic agent). If a distinct device, vacuum source 38 may be connected via a common delivery line with the therapeutic agent source 36 using a valve 40.

Turning now to FIGS. 2, 3, and 4, one exemplary use of the catheter 10 is shown and described. The catheter 10 may be advanced along a guidewire 26 within a vessel V such that the second balloon 12b in a deflated condition is adjacent to a treatment area, such as a lesion L. The second balloon 12b may then be expanded (note inflated balloon 12b′) and, in view of the non-compliant characteristics (note fibers F, which may be helical, longitudinal, or both), used to dilate or compact the lesion (note compacted lesion L′ in FIG. 4). Once compacted, the second balloon 12b may then be deflated, per FIG. 4.

To form a chamber C of suitable length to capture the lesion L, the outer portion or shaft 14a may be moved relative to the inner shaft 14b to adjust the relative spacing of the first and third balloons 12a, 12c (which balloons are shown as being elongated as just an example of a different shape), such as by releasing and then redeploying the retainer 29. For example, the outer portion or shaft 14a may be pulled back relative to the inner portion or shaft 14b to increase the spacing between the first and third balloons 12a, 12c, or may be advanced to decrease this spacing (see action arrow X in FIG. 1, indicating relative extension of retraction in the longitudinal direction). This ensures that the chamber C thus formed on inflation of the first and third balloons 12a, 12c corresponds to the margins of the treatment area, and thus is sized appropriately for treating the compacted lesion L therein. The adjustability afforded also avoids the need for maintaining a variety of catheters with different spacing of the balloons to accommodate a variety of lesion lengths.

Once in the desired position, the first and third balloons 12a, 12c may be expanded (note inflated balloons 12a′, 12c′). In view of the compliant or semi-compliant characteristics of these balloons 12a, 12c, inflation thus allows the outer surface of the balloons and, particularly to establish close contact with the interior wall I of the vessel V, thereby forming a seal. The therapeutic agent A may then be delivered to the now-sealed chamber C thus formed between the inflated balloons 12a′, 12c′, which includes the compacted lesion L′, and provide the intended pharmaceutical treatment effect.

The agent A may be provided for a set amount of time or a set volume, which may be selected by the clinician depending on the desired treatment regimen. The middle or second balloon 12b may also be expanded during this time to increase the pressure in the chamber Con the fluid therein including the agent A, thereby aiding in ensuring that intimate contact is made by the agent A with all parts of the compacted lesion L′. If not previously done, the middle or second balloon 12b could also be deployed (or redeployed) to compact the lesion L.

Thereafter, a vacuum may be applied to the dispersal lumen 30 associated with port 32 (such as via source 38) to withdraw any remaining therapeutic agent. The inflated balloons 12a, 12b, 12c may then be deflated, and the catheter 10 withdrawn from the vessel V, or repositioned for a further treatment, if desired.

In terms of materials, noncompliant (high-pressure) balloons are typically made of polyester or nylon. Semi-compliant (mid-pressure) balloons are commonly made of nylon, Pebax or higher-durometer polyurethanes. Compliant (elastomeric) balloons are typically made of polyurethane or silicone. However, the foregoing materials are simply examples, and the disclosed concepts are not considered to be limited to any particular materials or manner of fabrication. Summarizing, this disclosure may be considered to relate to the following items:
1. A medical apparatus for treating a lesion, comprising:
a shaft;
a first inflatable balloon supported by the shaft;
a second inflatable balloon supported by the shaft, a longitudinal position of the second inflatable balloon being adjustable relative to the first inflatable balloon; and
a third inflatable balloon supported by the shaft between the first inflatable balloon and the second inflatable balloon.
2. The medical apparatus of item 1, wherein the shaft comprises an outer shaft supporting the first inflatable balloon and an inner shaft supporting the second inflatable balloon.
3. The medical apparatus of item 2, wherein the inner shaft further supports the third inflatable balloon.
4. The medical apparatus of item 2 or 3, wherein the inner shaft comprises a first port proximal of the third inflatable balloon and a second port distal of the third inflatable balloon.
5. The medical apparatus of item 2 or 3 or 4, wherein the outer shaft comprises a retainer for retaining a position of the outer shaft relative to the inner shaft.
6. The medical apparatus of any of items 2-5, wherein the outer shaft comprises an first inflation port for the first inflatable balloon, and the inner shaft comprises a second inflation port for the second inflatable balloon and a third inflation port for the third inflatable balloon.
7. The medical apparatus of any of items 1-6, wherein a proximal end of the shaft comprises an infusion port for communicating with at least one opening in the shaft between the first inflatable balloon and the third inflatable balloon, and potentially a plurality of openings in the shaft between the first inflatable balloon and the third inflatable balloon.
8. The medical apparatus of any of items 1-7, wherein the first and second inflatable balloons are compliant or semi-compliant.
9. The medical apparatus of any of items 1-8, wherein the third inflatable balloon is elongated.
10. The medical apparatus of any of items 1-9, wherein the first and second inflatable balloons are generally spherical.
11. The medical apparatus of any of items 1-10, wherein the third inflatable balloon comprises a barrel section for compacting a lesion when inflated and tapered end sections connected to the shaft.
12. The medical apparatus of any of items 1-11, wherein the third inflatable balloon is non-compliant.
13. A medical apparatus for treating a lesion, comprising:
a shaft including telescoping inner and outer portions;
a first inflatable balloon supported by the inner portion of the shaft;
a second inflatable balloon supported by the outer portion of the shaft, a longitudinal position of the second inflatable balloon being adjustable relative to the first inflatable balloon by telescoping the outer portion of the shaft relative to the inner portion of the shaft; and
a third inflatable balloon supported by the inner shaft between the first inflatable balloon and the second inflatable balloon.
14. The medical apparatus of item 13, wherein the inner portion of the shaft comprises a first port proximal of the third inflatable balloon and a second port distal of the third inflatable balloon.
15. The medical apparatus of item 13 or item 14, wherein the outer portion of the shaft comprises a retainer for retaining the outer portion of the shaft relative to the inner portion of the shaft.
16. The medical apparatus of any of items 13-15, wherein the inner portion of the shaft comprises a first inflation port for the first inflatable balloon, the outer portion of the shaft comprises a second inflation port for the second inflatable balloon, and the inner portion of the shaft comprises a third inflation port for the third inflatable balloon.
17. The medical apparatus of any of items 13-16, wherein a proximal end of the shaft comprises a port for communicating with at least one opening in the shaft between the first and third inflatable balloons, and potentially a plurality of openings in the shaft between the first inflatable balloon and the third inflatable balloon.
18. The medical apparatus of any of items 13-17, wherein the first and second inflatable balloons are compliant or semi-compliant.
19. The medical apparatus of any of items 13-18, wherein the third inflatable balloon is elongated.
20. The medical apparatus of any of items 13-19, wherein the first and second inflatable balloons are generally spherical.
21. The medical apparatus of any of items 13-20, wherein the third inflatable balloon comprises a barrel section for compacting a lesion when inflated and tapered end sections connected to the shaft.
22. The medical apparatus of any of items 13-21, wherein the third inflatable balloon is non-compliant.
23. A medical apparatus for treating a lesion, comprising:
a shaft including telescoping inner and outer portions;
a first inflatable balloon supported by the inner portion of the shaft; and
a second inflatable balloon supported by the inner portion of the shaft.
24. The medical apparatus of item 23, wherein the inner portion of the shaft comprises a first port proximal of the second inflatable balloon and a second port distal of the second inflatable balloon.
25. The medical apparatus of item 23 or 24, wherein the outer portion of the shaft comprises a retainer for retaining the outer portion of the shaft relative to the inner portion of the shaft.
26. The medical apparatus of any of items 23-25, further including a third inflatable balloon supported by the outer shaft.
27. The medical apparatus of item 26, wherein the third inflatable balloon is located proximally of the first and second inflatable balloons.
28. The medical apparatus of item 26 or item 27, where the inner portion of the shaft comprises a first inflation port for the first inflatable balloon and a second inflation port for the second inflatable balloon, and the outer portion of the shaft comprises a third inflation port for the third inflatable balloon.
29. The medical apparatus of any of items 23-28, wherein a proximal end of the shaft comprises a port for communicating with at least one opening in the shaft between the first and second inflatable balloons, potentially a plurality of openings in the shaft between the first inflatable balloon and the second inflatable balloon.
30. The medical apparatus of any of items 23-29, wherein the first inflatable balloon is non-compliant and second inflatable balloons is compliant or semi-compliant.
31. The medical apparatus of any of items 23-30, wherein the first inflatable balloon comprises a barrel section for compacting a lesion when inflated and tapered end sections connected to the shaft.
32. A method for treating a lesion, comprising:
adjusting the position of a first inflatable balloon supported by a shaft relative to a second inflatable balloon supported by the shaft;
inflating a third inflatable balloon supported by the shaft between the first inflatable balloon and the second inflatable balloon.
33. The method of item 32, further including the step of delivering a therapeutic agent to a space between the first and second inflatable balloons prior to the step of inflating the third inflatable balloon.
34. The method of item 32, further including the step of delivering a therapeutic agent to a space between the first and second inflatable balloons after the step of deflating the third inflatable balloon.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, including +/−10% or less, +/−5% or less, +/−1% or less, and +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the spirit and scope of the appended claims. For example, the second or intermediate balloon 12b could remain non-deployed until after the agent A is delivered to the chamber C formed by the first and third balloons 12a, 12c. It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment may also be illustratively described and presented in the context or format of a plurality of separate embodiments.

1. A medical apparatus for treating a lesion, comprising:
a shaft;
a first inflatable balloon supported by the shaft;
a second inflatable balloon supported by the shaft, a longitudinal position of the second inflatable balloon being adjustable relative to the first inflatable balloon; and
a third inflatable balloon supported by the shaft between the first inflatable balloon and the second inflatable balloon.

2. The medical apparatus of claim 1, wherein the shaft comprises an outer shaft supporting the first inflatable balloon and an inner shaft supporting the second inflatable balloon.

3. The medical apparatus of claim 2, wherein the inner shaft further supports the third inflatable balloon.

4. The medical apparatus of claim 2, wherein the inner shaft comprises a first port proximal of the third inflatable balloon and a second port distal of the third inflatable balloon.

5. The medical apparatus of claim 2, wherein the outer shaft comprises a retainer for retaining a position of the outer shaft relative to the inner shaft.

6. The medical apparatus of claim 2, wherein the outer shaft comprises a first inflation port for the first inflatable balloon, and the inner shaft comprises a second inflation port for the second inflatable balloon and a third inflation port for the third inflatable balloon.

7. The medical apparatus of claim 1, wherein a proximal end of the shaft comprises an infusion port for communicating with at least one opening in the shaft between the first inflatable balloon and the third inflatable balloon.

8. The medical apparatus of claim 1, wherein:
the first and second inflatable balloons are compliant or semi-compliant;
the third inflatable balloon is elongated;
the first and second inflatable balloons are generally spherical.
the third inflatable balloon comprises a barrel section for compacting the lesion when inflated and tapered end sections connected to the shaft.
the third inflatable balloon is non-compliant

9-12. (cancelled)

13. A medical apparatus for treating a lesion, comprising:
a shaft including telescoping inner and outer portions;
a first inflatable balloon supported by the inner portion of the shaft;
a second inflatable balloon supported by the outer portion of the shaft, a longitudinal position of the second inflatable balloon being adjustable relative to the first inflatable balloon by telescoping the outer portion of the shaft relative to the inner portion of the shaft; and
a third inflatable balloon supported by the inner portion of the shaft between the first inflatable balloon and the second inflatable balloon.

14. The medical apparatus of claim 13, wherein the inner portion of the shaft comprises a first port proximal of the third inflatable balloon and a second port distal of the third inflatable balloon.

15. The medical apparatus of claim 13, wherein the outer portion of the shaft comprises a retainer for retaining the outer portion of the shaft relative to the inner portion of the shaft.

16. The medical apparatus of claim 13, wherein the inner portion of the shaft comprises a first inflation port for the first inflatable balloon, the outer portion of the shaft comprises a second inflation port for the second inflatable balloon, and the inner portion of the shaft comprises a third inflation port for the third inflatable balloon.

17. The medical apparatus of claim 13, wherein a proximal end of the shaft comprises a port for communicating with at least one opening in the shaft between the first and third inflatable balloons.

18. The medical apparatus of claim 13, wherein;
the first and second inflatable balloons are compliant or semi-compliant;
the third inflatable balloon is elongated;
the first and second inflatable balloons are generally spherical.
the third inflatable balloon comprises a barrel section for compacting the lesion when inflated and tapered end sections connected to the shaft.
the third inflatable balloon is non-compliant.

19.-22. (cancelled)

23. A medical apparatus for treating a lesion, comprising:
a shaft including telescoping inner and outer portions;
a first inflatable balloon supported by the inner portion of the shaft; and
a second inflatable balloon supported by the inner portion of the shaft.

24. The medical apparatus of claim 23, wherein the inner portion of the shaft comprises a first port proximal of the second inflatable balloon and a second port distal of the second inflatable balloon.

25. The medical apparatus of claim 23, wherein the outer portion of the shaft comprises a retainer for retaining the outer portion of the shaft relative to the inner portion of the shaft.

26. The medical apparatus of claim 23, further including a third inflatable balloon supported by the outer portion of the shaft.

27. The medical apparatus of claim 26, wherein the third inflatable balloon is located proximally of the first and second inflatable balloons.

28. The medical apparatus of claim 26, where the inner portion of the shaft comprises a first inflation port for the first inflatable balloon and a second inflation port for the second inflatable balloon, and the outer portion of the shaft comprises a third inflation port for the third inflatable balloon.

29. The medical apparatus of claim 23, wherein a proximal end of the shaft comprises a port for communicating with at least one opening in the shaft between the first and second inflatable balloons.

30. The medical apparatus of claim 23, wherein the first inflatable balloon is non-compliant and second inflatable balloon is compliant or semi-compliant, and the first inflatable balloon comprises a barrel section for compacting the lesion when inflated and tapered end sections connected to the shaft.

31.-34. (cancelled)
